(19) United States
(12) Patent Application Publication
(10) Pub. No.: US 2014/0261518 A1
(43) Pub. Date: Sep. 18, 2014

(54) FORMULATIONS AND METHODS FOR STRAIGHTENING AND REVITALIZING HAIR

(71) Applicant: ZOTOS INTERNATIONAL, INC., Darien, CT (US)

(72) Inventors: Andrew SAVAIDES, Norwalk, CT (US); Rushi TASKER, Trumbull, CT (US)

(73) Assignee: ZOTOS INTERNATIONAL, INC., Darien, CT (US)

(21) Appl. No.: 13/800,562

(22) Filed: Mar. 13, 2013

(51) Int. Cl. A61K 8/65; A61K 8/46; A45D 7/06; A61Q 5/06

(52) U.S. Cl. CPC A61K 8/65; A61Q 5/06; A61K 8/46; A45D 7/06
USPC 132/206; 424/70.2; 424/70.5; 424/70.51

(57) ABSTRACT

The present invention is directed to methods and formulations of treating keratin fibers, in order to provide long lasting curl reduction and styling results for keratin fibers from a single application. The present invention may include forming sufficient active sites on the keratin fibers followed with fixation and/or cross-linking of active compounds via covalent and strong ionic bond formation to the active sites on the keratin fibers. The active sites may be produced by breaking a sufficient number of disulfide bonds in the keratin fibers by using one or more reducing agents. The active compounds may then be fixed and/or cross-linked to the active sites. The fixation and/or cross-linking reactions of the active compounds onto the keratin fibers is facilitated by allowing the formulations containing the active compounds to react on the keratin fibers at ambient conditions for a sufficient time, followed with heating the keratin fibers to approximately 400° F.

### BACKGROUND OF THE INVENTION

1. Field of the Invention

The present invention is directed to methods and formulations of treating hair keratin fibers to provide long lasting curl reduction and styling results from a single application and treatment regardless of the frequency of number of shampoos and grooming techniques, and more particularly to methods and formulations configured for formation of sufficient active sites in hair followed with fixation and cross linking of active compounds via covalent and strong ionic bond formation.

2. Description of Related Art

Human hair is characterized as multi-textured from fine to coarse and curly to straight. The primary component of hair is keratin which is found in wools, nails, feathers, horns etc. Keratin is a fibrous protein that covers and protects the outer skin of living bodies. Keratin is comprised of 18 different amino acids including acidic, monobasic and dibasic and a high level of cystine. The integrity and resilience of keratin proteins is due to the highly cross linked structures with cystine. For example, hair keratin can have a cystine crosslinkage formed from an S—S bond per each 10-20 amino acid residues.

Due to the large affinity of keratin to hair, solubilized forms of keratin are added into hair cosmetic products to treat, repair and prevent hair damage from, grooming, environmental and chemical treatments. These are based on the adsorption of solubilized keratin into hair and desorption from subsequent shampooing. Quite often these efforts have a temporary effect of a few shampoos on the styling and manageability and other attributes of hair including shine, volume, hold suppleness, vitality or softness. This temporary effect is due to insufficient deposition and temporary fixation of keratin, and is referred as “under-keratinization.” However, the deposition of high levels of keratin into hair with a fixation that has long lasting results, creates an unbalanced and unstable condition of hair to respond in high humidity conditions. This condition is referred as “over-keratinization,” where hair becomes brittle and damaged, thus is susceptible and prone to breakage very easily during combing, brushing or other styling techniques.

During the past few years there is a new growing trend in the market for semi-permanent curl reduction and change in the styling of hair with minimum hair damage. These products affect the styling configuration of hair with discernible curl reduction and easiness of styling attributes including shine luster, smoothness, volume reduction and feel of hair. The art of temporary smoothing and removing curl has been practiced by applying pomades on hair followed by combing with uncontrollable hot heated metal combs. This technique had many severe drawbacks of scalp burning and excessive heat hair damage. Recently progress has been made by using controllable flat and curling irons for the straightening technique. However, the straightening and smoothing effects on hair are totally temporary, and total reversion occurs during perspiration, exposure to high humidity and especially after a single shampoo.

The technique of achieving these semi permanent results of straightening and smoothing was introduced by Brazilian stylists where they used solutions of formaldehyde from 0.2-1.5% to reduce curl with longevity of about four to six shampoos. This technique was known as “escova progressiva” where the hair is shampooed several times with high pH shampoos of about pH 8.5 to swell the hair, and then the “defrisse lotion” containing formaldehyde and thermal protectors was applied on the hair and processed for 20-30 minutes. The hair was then blown dried and flat ironed. The results from this Brazilian process resulted in temporary results of straight, silky, shiny and smooth hair. However, in order to attain semi-permanent results lasting beyond two to three shampoos this straightening process required weekly repeat applications.

Recently several products have entered the market labeled as “keratin treatments.” The composition of these products has one or more keratin cross linkers, solubilized keratin protein fractions, emollients, surfactants/emulsifiers and preservatives. The keratin cross linkers include monoaldehydes, dialdehydes and polyaldehydes at concentrations of 2%-10%. The chemical crosslinking and hardening of the proteins with the aldehydes is due to the Maillard reaction. The monoaldehydes are referred to as formal, methanal, or acetaldehyde. These aldehyde based keratin treatment products have many disadvantages. The major disadvantage with these aldehyde products is their toxicological profiles which signal safety and health concerns.

Formaldehyde also known as “formal,” “methanol,” or “methylene glycol” is a suspected carcinogen, and can cause severe contact dermatitis. Formaldehyde can cause immediate reactions to the immune system, and it is a cancer hazard. It is listed as a human carcinogen in the 12^{th} Report on Carcinogens published by the National Toxicology Program. Exposure to formaldehyde can be highly irritating to the eye, nose and throat, which can cause coughing and sneezing. Formaldehyde can cause severe allergic reactions of the skin, eyes, and respiratory tract, and long term exposure to low levels in the air can cause asthma-like respiratory problems and skin irritations such as dermatitis and itching. In women, exposure to formaldehyde can also cause menstrual disorders.

Formaldehyde is a colorless, strong-smelling and hazardous chemical that is found in hair smoothing treatments, including the Brazilian Blowout®, owned by Crème De Le Crème Inc. of West Hollywood, Calif. The Brazilian Blowout is regarded as being a more effective and less time-consuming choice than other hair-straightening methods, including conventional relaxers, Japanese thermal processing or keratin based treatments. Many stylists have gotten ill from repeated exposure to these treatments, for example as recently addressed by the Oregon OSHA agency. If hair salons do choose to use the hair straightening treatments that contain formaldehyde, they must comply with strict requirements set out in OSHA's formaldehyde standard, which sets a permissible exposure limit for formaldehyde in the workplace at 0.75 parts of formaldehyde per million parts of air (0.75 ppm). Furthermore, the standard requires that employers test the air to find out the level of formaldehyde present in the air when the product is being used.

Products containing >0.10% formaldehyde are prohibited in many country markets. Since these products are unstable they are formulated with a large excess of formaldehyde exceeding the permissible level. At levels of <2% formaldehyde, limited crosslinking and polymerization occurs on hair with some level of curl relaxation with shiny and better fiber alignment shown as frizz reduction. The curl reversion is almost quantitative within two to three shampoos but the cuticullar attributes have a few more shampoos of longevity. At higher concentration of formaldehyde, for example 4-8%, high crosslinking and fast rates of polymerization occur with a discernable curl reduction of hair. Also at these high levels there is no waiting period of 72 hours before shampooing, and hair can be even shampooed on the same or next day as the treatment.

Even though the hair appears shiny and healthy, the formaldehyde polymerization seals the cuticle and traps some of the formula agents into the hair shaft or cortex making the hair unhealthy. This is due to the water displacement and changes to the melanin, cortical cells and matrix of hair. Over time the changes in the cortical cells and microfilaments are irreversible and result in hair damage. Repeat treatments can amplify this damage which results in fiber failures and hair breakage.

Due to the safety issues of formaldehyde and hair damage, what is needed is a safe system to treat and straighten hair with acceptable longevity of product effects on hair including curl, volume reduction, smoothness, shine, luster, glossiness and ease of manageability. Furthermore is desirable to have a semi-permanent system that would have gradual reversion of the curl, volume, and smoothening for up to 90 days or approximately 48-50 shampoos.

### SUMMARY OF THE INVENTION

The present invention is designed to overcome the above noted limitations that are attendant upon the use of conventional hair treatment and straightening systems and, toward this end, it contemplates the provision of novel methods and formulations of treating hair keratin fibers to provide long lasting curl reduction and styling results from a single application and treatment regardless of the frequency of number of shampoos and grooming techniques.

Exemplary embodiments of the present invention may be directed to methods and formulations of treating keratin fibers, in order to provide long lasting curl reduction and styling results for keratin fibers from a single application. The present invention may include forming sufficient active sites on the keratin fibers followed with fixation and/or cross-linking of active compounds via covalent and strong ionic bond formation to the active sites on the keratin fibers. The active sites may be produced by breaking a sufficient number of disulfide bonds in the keratin fibers by using one or more reducing agents. The active compounds may then be fixed and/or cross-linked to the active sites. The fixation and/or cross-linking reactions of the active compounds onto the keratin fibers is facilitated by allowing the formulations containing the active compounds to react on the keratin fibers at ambient conditions for a sufficient time, followed with heating the keratin fibers to approximately 400° F.

It is accordingly an object of the present invention to provide treatments for reducing curls and/or improving styling results that can safely be applied to the hair.

It is another object of the present invention to provide treatments for reducing curls and/or improving styling results that are non-damaging to the hair.

It is yet another object of the present invention to provide treatments for reducing curls and/or improving styling results that provide long lasting results even after repeated styling and/or cleansing of the hair.

It is still another object of the present invention to provide treatments for reducing curls and/or improving styling results that revitalizes and/or improves the quality of the hair.

It is yet another object of the present invention to provide treatments for reducing curls and/or improving styling results that are configured to form sufficient active sites in hair, followed with fixation and cross linking of active compounds via covalent and strong ionic bond formation.

In accordance with these objects, the present invention contemplates an exemplary embodiment of the present invention of a method of treating keratin fibers may include applying a softening composition including at least one reducing agent to the keratin fibers, applying a keratin mixture including at least one hydrolyzed keratin and at least one oxidizing agent to the keratin fibers, and heating the keratin fibers to at least 400° F.

According to this exemplary embodiment of the present invention, the method may also include shampooing the keratin fibers and blotting the keratin fibers dry with a towel.

According to this exemplary embodiment of the present invention, a leave in condition and/or deep rinse off condition may be applied to the keratin fibers and then the keratin fibers may be rinsed with water for approximately 5 to 7 minutes.

According to this exemplary embodiment of the present invention, applying the softening composition may include applying the softening composition onto the keratin fibers with a brush while maintaining the keratin fibers straight.

According to this exemplary embodiment of the present invention, the at least one reducing agent may include Thioglycolic acid, Thiolactic acid, 3-mercaptoproprionic acid, Thiomalic acid, Glyceryl Mono Thioglycolate, Cysteine, Thioglycerol (3-Mercaptopropane-1,2-diol), Cysteamine, sulfites, bisulfites and/or combinations thereof.

According to this exemplary embodiment of the present invention, the at least one reducing agent may be about 6-16% of the softening composition based on the entire weight of the softening composition.

According to this exemplary embodiment of the present invention, the keratin mixture may be made of approximately one part keratin concentrate composition comprising the at least one hydrolyzed keratin and approximately three parts keratin sealer composition comprising the at least one oxidizing agent.

According to this exemplary embodiment of the present invention, the keratin concentrate composition may also include about 0.1-0.5% of at least one thickening agent, about 2-15% of at least one quaternized hydrolyzed keratin, and about 1-6% at least one silicone cystine and/or silicone cysteine copolymer based on the entire weight of the keratin concentrate composition.

According to this exemplary embodiment of the present invention, the at least one hydrolyzed keratin comprises about 4-30% of the keratin concentrate composition based on the entire weight of the keratin concentrate composition.

According to this exemplary embodiment of the present invention, the oxidizing agent may be hydrogen peroxide, potassium perborate, potassium bromated and/or combinations thereof.

According to this exemplary embodiment of the present invention, the keratin fibers that are treated include hair.

According to this exemplary embodiment of the present invention, the softening composition may be applied to the keratin fibers for 10 to 30 minutes.

According to this exemplary embodiment of the present invention, the method may also include rinsing at least part of the hair softening composition from the keratin fibers prior to applying the keratin mixture.

In accordance with these objects, the present invention contemplates an exemplary embodiment of the present invention of a kit for treating keratin fibers that may include a softening composition comprising at least one reducing agent, a keratin concentrate composition comprising at least one hydrolyzed keratin, and a keratin sealer composition comprising at least one oxidizing agent.

According to this exemplary embodiment of the present invention, the keratin concentrate composition is configured for mixing with the keratin sealer composition to form a keratin mixture.

According to this exemplary embodiment of the present invention, the at least one reducing agent may be Thioglycolic acid, Thiolactic acid, 3-mercaptoproprionic acid, Thiomalic acid, Glyceryl Mono Thioglycolate, Cysteine, Thioglycerol (3-Mercaptopropane-1,2-diol), Cysteamine, sulfites, bisulfites and/or combinations thereof.

According to this exemplary embodiment of the present invention, the at least one reducing agent may be approximately 6-16% of the softening composition based on the entire weight of the softening composition.

According to this exemplary embodiment of the present invention, the keratin mixture is made of approximately one part of the keratin concentrate composition and approximately three parts of the keratin sealer composition.

According to this exemplary embodiment of the present invention, the keratin concentrate composition further comprises about 0.1-0.5% of at least one thickening agent, about 2-15% of at least one quaternized hydrolyzed keratin, and about 1-6% at least one silicone cystine and/or silicone cysteine copolymer based on the entire weight of the keratin concentrate composition.

According to this exemplary embodiment of the present invention, the at least one hydrolyzed keratin may be about 4-30% of the keratin concentrate composition based on the entire weight of the keratin concentrate composition.

According to this exemplary embodiment of the present invention, the at least one hydrolyzed keratin may include at least one quaternized hydrolyzed keratin.

According to this exemplary embodiment of the present invention, the oxidizing agent comprises hydrogen peroxide, potassium perborate, potassium bromated or combinations thereof.

According to this exemplary embodiment of the present invention, the at least one silicone cystine and/or silicon cysteine copolymer comprises cystine bis-PG-propyl silanetriol.

According to this exemplary embodiment of the present invention, the softening composition is configured to form at least one active site on the keratin fibers, and wherein the keratin mixture is configured to fix or cross-link the at least one hydrolyzed keratin to the at least one active site.

According to this exemplary embodiment of the present invention, the reducing agent comprises at least one acylated derivative of Cysteine, Thioglycerol or Cysteamine.

### BRIEF DESCRIPTION OF THE SEVERAL VIEWS OF THE DRAWINGS

For a fuller understanding of the nature and object of the present invention, reference should be had to the following detailed description taken in connection with the accompanying drawings, in which:

FIG. 1 is a graph showing the styling time required for untreated hair in comparison to the styling time required for hair treated in accordance with an exemplary method and formulation according to the present invention;

FIG. 2 is a graph showing percentage of curl level for untreated hair in comparison to the percentage of curl level for hair treated in accordance with an exemplary method and formulation according to the present invention;

FIG. 3 is a graph showing percentage of frizz level for untreated hair in comparison to the percentage of frizz level for hair treated in accordance with an exemplary method and formulation according to the present invention;

FIG. 4 is a graph showing percentage of shine, luster and sheen level for untreated hair in comparison to the percentage of shine, luster and sheen level for hair treated in accordance with an exemplary method and formulation according to the present invention;

FIG. 5 is a chart showing the overall smoothing and revitalizing effects of hair treated in accordance with an exemplary method and formulation according to the present invention;

FIG. 6A is a photo showing the untreated hair of an exemplary consumer panelist;

FIGS. 6B and 6C are photos showing the treated hair of the exemplary consumer panelist from FIG. 6A treated in accordance with an exemplary method and formulation according to the present invention;

FIG. 7A is a photo showing the untreated hair of an exemplary consumer panelist;

FIGS. 7B and 7C are photos showing the treated hair of the exemplary consumer panelist from FIG. 7A treated in accordance with an exemplary method and formulation according to the present invention;

FIG. 8A is a photo showing the untreated hair of an exemplary consumer panelist; and

FIGS. 8B and 8C are photos showing the treated hair of the exemplary consumer panelist from FIG. 8A treated in accordance with an exemplary method and formulation according to the present invention.

### DESCRIPTION OF THE INVENTION

The present invention now will be described more fully hereinafter with reference to the accompanying figures. The invention maybe embodied in many different forms and should not be construed as limited to the embodiments set forth herein.

Exemplary embodiments of the present invention are directed to methods and formulations of treating keratin fibers, for example keratin fibers comprising hair, in order to provide long lasting curl reduction and styling results for keratin fibers from a single application and treatment regardless of the frequency of number of shampoos and grooming techniques applied to the keratin fibers after the treatment. Accordingly, an exemplary method of the present invention includes forming sufficient active sites on the keratin fibers, for example hair, followed with fixation and/or cross-linking of active compounds via covalent and strong ionic bond formation to the active sites on the keratin fibers, and exemplary formulations of the present invention include formulations configured to form active sites on keratin fibers and formulations configured to cause fixation and/or cross-linking of active compounds to the active sites on the keratin fibers. The active sites may be produced by breaking a sufficient number of disulfide bonds in the keratin fibers by using one or more reducing agents, such as but not limited to, Thioglycolic acid (TGA), Thiolactic acid (TLA), 3-mercaptoproprionic acid, Thiomalic acid, Glyceryl Mono Thioglycolate (GMT), esters and salts of these exemplary acids, Cysteine, Thioglycerol (3-Mercaptopropane-1,2-diol), Cysteamine, and their acylated derivatives, sulfites, such as sodium sulfite, potassium sulfite, lithium sulfite calcium sulfite and guanidine sulfite, bisulfites, such as ammonium bisulfite, monoethanolamine bisulfite, diethanolamine bisulfite, isopropanolamine bisulfite, diisopropanolamine bisulfite, sodium bisulfite, potassium bisulfite, lithium bisulfite, calcium bisulfite, guanidine bisulfite, sodium metabisulfite, potassium metabisulfite, lithium metabisulfite, calcium metabisulfite and guanidine metabisulfite, and combinations thereof.

The active sites produced may include, but are not limited to, Sulfhydryl (K—SH), Amino (K—NH2, K—NH—K) and/or Carboxyl (K—COOH), and exemplary formulations containing active compounds that may be fixed and/or cross-linked to the active sites include mixtures of soluble keratin protein mixtures that may containing high levels of cystine, and an oxidant which may be left on the keratin fibers for about 24 hours. The fixation and/or cross-linking reactions of the active compounds onto the keratin fibers, such as hair, is facilitated by allowing the exemplary formulations containing the active compounds to react on the keratin fibers at ambient conditions for a sufficient time, for example approximately 15 minutes, followed with heating the keratin fibers to approximately 400° F.

According to exemplary embodiments of the present invention, straight, curly or frizzy hair of different textures may be treated in accordance with exemplary embodiments of the present invention order to provide long lasting curl reduction and styling results for the hair. In order to treat the hair according to an exemplary embodiment of the present invention, the hair to be treated is shampooed only once, and towel blotted dry. For damaged hair, a leave in conditioner may be applied or a deep rinse off conditioner may be applied for 5-7 minutes followed with water rinsing. Then a hair softening composition that is configured to form active sites on the keratin fibers of the hair, is applied on the hair in small sections with a brush while combing and maintaining the hair straight and processed for approximately 10-30 minutes at ambient temperatures depending on the texture of the hair. The hair softening composition may preferably be in the form of a viscous liquid, and may have exemplary formulations as show in Table 1A, Table 1B, Table 1C and Table 1D. It is understood that the hair softening composition may also have other formulations and/or forms in accordance with the present invention, and for example may be in the form of a gel, cream, lotion or spray.

TABLE 1A

| HAIR SOFTENING COMPOSITION | w/w % |
|---|---|
| Water | QS to 100 |
| Polyquaternium-10 | 0.005-1 |
| Steareth-2 | 1-5 |
| Steareth-10 | 1-5 |
| Stearyl Alcohol (and) Ceteareth-20 | 5-10 |
| Mineral Oil | 2-10 |
| Petrolatum | 2-4 |
| Cetyl Alcohol | 4-8 |
| Propylene Glycol | 1-3 |
| Fragrance (Parfum) | 0.5-1 |
| Soyethyl Morpholinium Ethosulfate | 1-3 |
| Glycine | 0.5-5 |
| Chelating agent | 0.2-1 |
| Reducing agent | 6-10 |
| Alkalizing agent | QS to pH |
| Buffering agent | QS to pH |

TABLE 1B

| HAIR SOFTENING COMPOSITION | w/w % |
|---|---|
| Water | QS to 100 |
| Glycerin | 0.5-1.50 |
| Ammonium Laureth Sulfate | 0.1-1.00 |
| Cetearyl Alcohol | 1.5-2.50 |
| Cetyl Alcohol | 0.5-1.50 |
| Steareth-21 | 0.5-1.50 |
| Steareth-2 | 0.5-1.50 |
| Stearic Acid | 0.5-1.50 |
| Hydrolyzed Wheat Protein | 0.01-0.60 |
| Fragrance | 0.5-2.00 |
| Polyquaternium-22 | 0.5-1.50 |
| Amodimethicone | 2.0-3.0 |
| Chelating agent | 0.2-1 |
| Reducing agent | 6-8 |
| Alkalizing agent | QS to pH |
| Buffering agent | QS to pH |

TABLE 1C

| HAIR SOFTENING COMPOSITION | w/w % |
|---|---|
| Water | QS to 100 |
| Sucrose | 6-8 |
| Acrylates Copolymer | 5.50-7.50 |
| Laureth-23 | 3-5 |
| Fragrance (Parfum) | 0.15-1.15 |
| PEG-12 Dimethicone | 0.5-2 |
| Chelating agent | 0.01-1 |
| Reducing agents | 12-20 |
| Alkalizing agent | QS to pH |
| Buffering agent | QS to pH |

TABLE 1D

| HAIR SOFTENING COMPOSITION | w/w % |
|---|---|
| Water | QS to 100 |
| Polyacrylate Crosspolymer-6 | 0.01-1.5 |
| Cetearyl Alcohol | 1-3 |
| Cetyl Alcohol | 0.5-1.5 |
| Steareth-21 | 0.25-1.5 |
| Steareth-2 | 0.5-1.5 |
| Stearic Acid | 0.25-1.5 |
| Propylene Glycol | 1-3 |
| Urea | 2-4 |
| Isopropyl Alcohol | 0.1-2 |
| Sucrose | 0.01-1.5 |
| Sodium Polyacrylate (and) Hydrogenated Polydecene | 0.25-1.5 |
| Fragrance/Parfum | 0.01-1.5 |
| Chelating agent | 0.01-1.5 |
| Reducing agent | 12-18 |
| Alkalizing agent | QS to pH |
| Buffering agent | QS to pH |

The chelating agents that may be used in the hair softening composition include, but are not limited to Editronic acid (HEDP), Tetrasodium HEDP, Trisodium HEDP, EDTA, Monosodium EDTA, Disodium EDTA, Trisodium EDTA, Tetrasodium EDTA, Monosodium HEDTA, Disodium HEDTA, Trisodium HEDTA, Tetrasodium HEDTA, Pentasodium Pentate and combinations thereof. The reducing agents that may be used in the hair softening composition include, but are not limited to Thioglycolic acid (TGA), Thiolactic acid (TLA), 3-mercaptoproprionic acid, Thiomalic acid, Glyceryl Mono Thioglycolate (GMT), esters and salts of these exemplary acids, Cysteine, Thioglycerol (3-Mercaptopropane-1,2-diol), Cysteamine, and their acylated derivatives, sulfites, such as sodium sulfite, potassium sulfite, lithium sulfite calcium sulfite and guanidine sulfite, bisulfites, such as ammonium bisulfite, monoethanolamine bisulfite, diethanolamine bisulfite, isopropanolamine bisulfite, diisopropanolamine bisulfite, sodium bisulfite, potassium bisulfite, lithium bisulfite, calcium bisulfite, guanidine bisulfite, sodium metabisulfite, potassium metabisulfite, lithium metabisulfite, calcium metabisulfite and guanidine metabisulfite, and combinations thereof.

The alkalizing agents that may be used in the hair softening composition include, but are not limited to, ammonia, monoethanol amine, diethanolamine, isopropanolamine, diisopropanolamine and combinations thereof. Buffering agents that may be used in the hair softening composition may include bicarbonates, such as ammonium bicarbonate, borates, such as sodium borate, citrates, such as ammonium citrate or phosphates.

The pH of the hair softening composition may be adjusted to 6-9 depending on the reducing agent or agents that are included in the hair softening composition. For example, the hair softening compositions shown in Tables 1A, 1B and 1C may preferably have a pH between about 8.1 and 8.5, and the hair softening composition shown in Table 1D may preferably have a pH between about 6.5 and 7.0. It is understood that one or more reducing agent included in the hair softening composition is configured to produce the active sites on the keratin fibers of the hair by breaking a sufficient number of disulfide bonds in the keratin fibers of the hair, as shown for example by Equation 1.

Formation of Cysteine Reactive Groups in Hair with a RA (Reducing Agent)

Hair-K1-S−S—K2-Hair+RA→Hair-K1-SH+Hair-K2-SH (Reduced Hair) (1)

After the hair softening composition has been applied to the hair and allowed to process, the hair is then rinsed with luke-warm water, blotted dry and combed carefully. A formulation containing active compounds to be fixed and/or cross-linked to the active sites formed on the keratin fibers of the hair may then be applied to the hair. The formulation containing the active compounds is a keratin mixture comprised of a keratin concentrate composition and a keratin sealer composition, preferably the keratin mixture is a mixture of the keratin concentrate composition and the keratin sealer composition mixed at a weight ration of 1:3. Other auxiliary ingredients may also be included in the keratin mixture. The keratin concentrate composition may have an exemplary formulation as shown in Table 2. It is understood that the keratin concentrate composition may also have other formulations and/or forms in accordance with the present invention, and for example may be in the form of a gel, cream, lotion or spray.

TABLE 2

| KERATIN CONCENTRATE COMPOSITION | w/w % |
|---|---|
| Thickening Agent | 0.1-0.5 |
| Hydrolyzed Keratin | 2-25 |
| Quaternized Hydrolyzed Keratin | 2-15 |
| Cystine Bis-PG-Propyl Silanetriol | 1-6 |
| Phenoxyethanol | 0.5-2 |
| Potassium Sorbate | 0.5-2 |
| Citric Acid | QS to pH 4-7 |
| Water | QS to 100 |

The thickening agents that may be used in the keratin concentrate composition include, but are not limited to, guar gum, hydroxylpropyl guar, sclerotium gum, hydroxy propyltrimonium chloride, xanthum gum and hydroxyethyl cellulose. The hydrolyzed keratin may preferably have about 5% of cystine, and an average molecular weight of 1000 to about 100,000 Daltons. The hydrolyzed keratin may be made up of a combination of hydrolyzed low molecular weight (Low MW) keratins and hydrolyzed high molecular weight (High MW) keratins. The Low MW keratins may be comprised of peptides of few amino acid residues, and have molecular weights of less than 40 kD, and their effect is more to the internal areas (cortex, matrix) of the hair since they penetrate the hair easier. The High MW keratins may be comprised of peptides with many amino acid residues, and have molecular weights of more than 100,000 kD, and their effect is to form strong films on the external (cuticle, cell membrane complex) areas of the hair.

It is understood that other silicone cystine/cysteine copolymers having a cystine or cysteine terminal group in the chain besides cystine bis-PG-propyl silanetriol may also be used in the keratin concentrate composition. It is understood that quanternized hydrolyzed keratin may include keratins that are cationic and carry a positive charge, for example cocodimonium hydroxylpropyl hydrolyzed keratin. The pH of the keratin concentrate composition may be 4-7, and more preferably pH 6.

The keratin sealer composition used in the keratin mixture may have an exemplary composition as shown in Table 3. It is understood that the keratin sealer composition may also have other formulations and/or forms in accordance with the present invention, and for example may be in the form of a gel, cream, lotion or spray.

TABLE 3

| KERATIN SEALER COMPOSITION | w/w % |
|---|---|
| Methylparaben | 0.05-0.10 |
| Oxidizing Agent | 1.33-2.66 |
| Phosphoric Acid | QS to pH 3.0-4.0 |
| Disodium Phosphate | 0-0.40 |
| Pentasodium Pentetate | 0.13-0.25 |
| Acetamidopropyl Trimonium Chloride | 0.67-1.00 |
| Polyquaternium-7 | 2.00-6.00 |
| Polyquaternium-6 | 0.60-1.50 |
| Cetrimonium Chloride | 0.10-0.20 |
| Propylene Glycol | 0.10-0.20 |
| PPG-26-Buteth-26 (and) PEG-40 Hydrogenated | 0.27-0.60 |
| Castor Oil | |
| VP/DMAPA Acrylates Copolymer | 0.25-0.60 |
| Silicone Quaternium-16 | 2.00-6.00 |
| Amodimethicone | 2.00-6.00 |
| Water | QS to 100 |

The oxidizing agent may be, but not limited to, hydrogen peroxide, potassium perborate, potassium bromate or combinations thereof. The keratin mixture of the keratin concentrate composition and keratin sealer composition may then be applied to the hair and processed for approximately 15 minutes. The hair may then be combed, and the excess keratin mixture is blotted. The hair may then be dried to about 80-90% with a blow dryer and a paddle brush at low temperature setting. The hair is then flat ironed at a 400-450° F. setting depending on the texture of the hair. The hair may then be shampooed and conditioned after 24 hours and styled as desired.

It is understood that a smoothing kit comprised of a hair softening composition, keratin concentrate composition and keratin sealer composition may be provided in accordance with exemplary embodiments of the present invention. It is further understood that the smoothing kit is not limited to any particular combination of hair softening compositions, keratin concentrate compositions and keratin sealer compositions discussed with respect to the present invention.

It is understood that according to other exemplary embodiments of the present invention the process can be modified when only hair nourishing or fizz reduction is desired, where the processing time or contact with hair of the hair softening composition can be reduced to about 5-10 minutes for normal resistant hair and about 3-5 minutes for color treated hair.

It is understood that the hair treatment according to the exemplary embodiment of the present invention of using the hair softening composition followed by application of the keratin mixtures provides a reduction/oxidation process that forms covalent bonds between the keratin fibers of the treated hair keratin with the hydrolyzed, partially hydrolyzed keratin and/or silicone cystine/cysteine copolymer of the keratin mixture. Exemplary reactions of the keratin fibers of the treated hair with the keratin mixture containing the hydrolyzed keratin proteins and/or the silicone cystine/cysteine copolymers are shown in Equations 2A-2D and 3A-3E.

### Crosslinking Reactions of the Hydrolyzed Keratin Proteins (HKP) and a Silicone Cystine Cross Polymer (SiCP)

Hair-K1-SH+HKP—K3-S−S—K4-HKP→Hair-K1-S−S—K4-HKP+HKP—K3-SH (2A)

Hair-K1-SH+HKP—K3-S−S—K4-HKP→Hair-K1-S−S—K3-HKP+HKP—K4-SH (2B)

Hair-K1-SH+CP-Si1-S−S—Si2-CP→Hair-K1-S−S—Si1-CP+HS—Si2-CP (2C)

Hair-K1-SH+CP-Si1-S−S—Si2-CP→Hair-K1-S−S—Si2-CP+HS—Si1-CP (2D)

Hair-K1-SH+HKP—K3-SH+H₂0₂→Hair-K1-S−S—K3-HKP (3A)

Hair-K2-SH+HKP—K3-SH+H₂0₂→Hair-K2-S−S—K3-HKP (3B)

Hair-K1-SH+HKP—K3-SH+H₂0₂→Hair-K1-S−S—K3-HKP (3C)

Hair-K2-SH+HS—Si2-CP+H₂0₂→Hair-K2-S−S—Si2-CP (3D)

Hair-K1-SH+HS—Si2-CP+H₂0₂→Hair-K1-S−S—Si2-CP (3E)

The degree of fixation and types of the active compounds, such as keratins, deposited onto the active sites of the keratin fibers of the treated hair change the quality of the treated hair, including attributes such as shine, vitality, volume, movement or softness. The fixation of these active compounds onto the keratin fibers of the treated hair is not by adsorption, but due to chemical intermolecular reactions and formation of covalent bonds with the nucleophilic active sites generated by the reducing agent. The degree of reaction of the active compounds and the nucleophilic generated active sites will determine the longevity of the treatment.

Other methods of fixation of the active compounds contemplated by the present invention include adding the active compounds, such as keratins, into a reducing composition, such as the hair softening composition, and thereby permitting simultaneous reactions between the active compounds and the nucleophilic generated active sites to take place simultaneously before neutralization. Exemplary reactions in accordance with this aspect of the present invention are shown in Equations 4A-4C.

### Crosslinking Reactions of the Hydrolyzed Keratin Proteins (HKP) and Silicone Cystine Cross Polymer of Unreduced Hair Via Sulfur-Sulfur Interchange

Hair-K1-S−S—K2-Hair+HKP—K3-S−S—K4-HKP→Hair-K1-S−S—K4-HKP+Hair-K2-S−S—K3-HKP (4A)

Hair-K1-S−S—K2-Hair+PC-Si1-S−S—Si2-CP→Hair-K1-S−S—Si2-CP+Hair-K2-S−S—Si1-CP (4B)

HKP—K3-S−S—K4-HKP+PC-Si1-S−S—Si2-CP→HKP—K3-S−S—Si2-CP+PC—Si1−S−S—K4-HKP (4C)

### EXAMPLES

The treatment method and composition examples are given below to further illustrate the exemplary embodiments of the present invention.

### Example 1

The Example Formulations 1, 2, 3 and 4 listed in the tables below, were evaluated on twenty clients with normal and color treated hair with various different texture types, density, curl pattern and length. All clients were serviced in accordance with the method of the present invention described above, where Example Formulation 1 of the hair softening composition was used on normal curly, frizzy hair, and Example Formulation 2 of the hair softening composition was used on colored curly hair up to 20 volume. The keratin mixture was prepared by mixing one part Example Formulation 3 and three parts Example Formulation 4, and then applied on to the hair, and processed in accordance with the method of the present invention described above.

All performance attributes of hair curl, frizz, volume reduction, and conditioning, feel, shine, and softness/smoothness were evaluated after the treatment. The straightening was very satisfactory and ranged from 60% curl reduction on very tight kinky curly hair, to 90% or more on wavy hair. The treated hair appeared lighter and healthier. The condition of the treated hair was also improved with higher shine, luster, better touch feel and healthy movement. All models expressed that their hair felt silky, lighter and thinner. The performance attributes of these models were monitored after the initial treatment day and then for three months at interval of every 3-4 weeks. The initial performance attributes related to smoothness and curl reduction may have been reduced after three weeks but the hair still maintained good shine, luster, less frizz with considerable less curl and healthy movement. Models also have expressed their styling time had been reduced to about 50%. Evaluation of the models treated hair after three months showed that the products still had noticeable smoothing and curl reduction effects on treated hair. Based on these observations the single treatment imparts long lasting smoothing and straightening effects for at least 90 days or three months on various hair texture types.

EXAMPLE FORMULATION 1

| HAIR SOFTENING COMPOSITION FOR | |
|---|---|
| NORMAL/RESISTANT HAIR | w/w % |
| Water | 56.075 |
| Polyquaternium-10 | 0.005 |
| Steareth-2 | 1.18 |
| Steareth-10 | 1.11 |
| Stearyl Alcohol (and) Ceteareth-20 | 6.31 |
| Mineral Oil | 2.1 |
| Petrolatum | 2.1 |
| Cetyl Alcohol | 4.2 |
| Propylene Glycol | 1.74 |
| Fragrance | 1.1 |
| Soyethyl Morpholinium Ethosulfate | 1 |
| Glycine | 0.5 |
| Pentasodium Pentetate | 0.2 |
| Cysteamine HCl (75%) | 12.88 |
| Isopropanolamine | 3.5 |

The pH of Example Formulation 1 is preferably 8.1-8.5.

EXAMPLE FORMULATION 2

| HAIR SOFTENING COMPOSITION FOR | |
|---|---|
| COLOR TREATED HAIR | w/w % |
| Water | 56.075 |
| Polyquaternium-10 | 0.005 |
| Steareth-2 | 1.18 |
| Steareth-10 | 1.11 |
| Stearyl Alcohol (and) Ceteareth-20 | 6.31 |
| Mineral Oil | 2.1 |
| Petrolatum | 2.1 |
| Cetyl Alcohol | 4.2 |
| Propylene Glycol | 1.74 |
| Fragrance | 1.1 |
| Soyethyl Morpholinium Ethosulfate | 1 |
| Glycine | 0.5 |
| Pentasodium Pentetate | 0.2 |
| Cysteamine HCl (75%) | 12.88 |
| Isopropanolamine | 3.0 |

The pH of Example Formulation 2 is preferably approximately 8.1-8.5.

EXAMPLE FORMULATION 3

| KERATIN CONCENTRATE COMPOSITION | w/w % |
|---|---|
| Water | 69.70 |
| Sclerotium Gum | 0.2 |
| Hydrolyzed Keratin Low MW (25%) | 4.8 |
| Hydrolyzed Keratin High MW (32%) | 19.2 |
| Cystine Bis-PG-Propyl Silanetriol | 4 |
| Phenoxyethanol | 1 |
| Potassium Sorbate | 1 |
| Citric Acid | 0.1 |

The pH of Example Formulation 3 is preferably approximately 6.5-7.50.

EXAMPLE FORMULATION 4

| KERATIN SEALER COMPOSITION | w/w % |
|---|---|
| Water | 86.37 |
| Methylparaben | 0.05 |
| Hydrogen Peroxide | 2.66 |
| Phosphoric Acid | 0.09 |
| Disodium Phosphate | 0.002 |
| Pentasodium Pentetate | 0.13 |
| Acetamidopropyl Trimonium Chloride | 0.67 |
| Polyquaternium-7 | 2.00 |
| Polyquaternium-6 | 0.67 |
| Cetrimonium Chloride | 0.13 |
| Propylene Glycol | 0.10 |
| PPG-26-Buteth-26 (and) PEG-40 Hydrogenated Castor Oil | 0.27 |
| VP/DMAPA Acrylates Copolymer | 0.25 |
| Silicone Quaternium-16 | 2.00 |
| Amodimethicone | 2.00 |

The pH of Example Formulation 4 is preferably approximately 3-4.

COMBINATION OF
EXAMPLE FORMULATIONS 3 & 4 (1:3)

| KERATIN MIXTURE | w/w % |
|---|---|
| Water | 83.96 |
| Sclerotium Gum | 0.05 |
| Hydrolyzed Keratin Low MW (25%) | 1.20 |
| Hydrolyzed Keratin High MW (32%) | 4.80 |
| Cystine Bis-PG-Propyl Silanetriol | 1.00 |
| Phenoxyethanol | 0.25 |
| Potassium Sorbate | 0.25 |
| Citric Acid | 0.025 |
| Methylparaben | 0.038 |
| Hydrogen Peroxide | 1.00 |
| Phosphoric Acid | 0.07 |
| Disodium Phosphate | 0.002 |
| Pentasodium Pentetate | 0.10 |
| Acetamidopropyl Trimonium Chloride | 0.50 |
| Polyquaternium-7 | 1.50 |
| PPG-26-Buteth-26 and PEG-40-Hydrogenated Castor oil | 0.20 |
| Fragrance | 0.20 |
| Polyquaternium-6 | 0.50 |
| Cetrimonium Chloride | 0.10 |
| Propylene Glycol | 0.08 |
| PPG-26-Buteth-26 | 0.20 |
| VP/DMAPA Acrylates Copolymer | 0.19 |
| Silicone Quaternium-16 | 1.50 |
| Amodimethicone | 1.50 |

The pH of the exemplary keratin mixture shown above is preferably 5-6.

### Example 2

The treatments and formulations according to exemplary embodiments of the present invention are not limited to only professional use by stylists, but may also be used for consumer home use. Formulations of hair softening compositions as shown in Example Formulation 5 for normal/resistant hair, and Example Formulation 6 for color treated hair were used in combination with a keratin mixture formed from Example Formulation 3 of the keratin concentrate and Example Formulation 4 of the keratin sealer and evaluated. Directions and products kits were provided to sixty consumers with different hair textures, density, curl pattern and length for evaluation.

EXAMPLE FORMULATION 5

| HAIR SOFTENING COMPOSITION FOR | |
|---|---|
| NORMAL/RESISTANT HAIR | w/w % |
| Water | QS to 100.00 |
| Glycerin | 1.00 |
| Ammonium Laureth Sulfate | 0.50 |
| Cetearyl Alcohol | 2.00 |
| Cetyl Alcohol | 1.00 |
| Steareth-21 | 0.75 |
| Steareth-2 | 1.00 |
| Stearic Acid | 0.75 |
| Hydrolyzed Wheat Protein | 0.10 |
| Fragrance | 1.1 |
| Polyquaternium-22 | 1.0 |
| Amodimethicone | 2.50 |
| Tetrasodium Etidronate | 0.36 |
| Thioglycolic acid | 7.56 |
| Isopropanolamine | 4.00 |
| Ammonium Bicarbonate | 2.50 |

The pH of Example Formulation 5 is preferably approximately 8.90.

EXAMPLE FORMULATION 6

| HAIR SOFTENING COMPOSITION FOR | |
|---|---|
| COLOR TREATED HAIR | w/w % |
| Water | QS to 100.00 |
| Glycerin | 1.00 |
| Ammonium Laureth Sulfate | 0.50 |
| Cetearyl Alcohol | 2.00 |
| Cetyl Alcohol | 1.00 |
| Steareth-21 | 0.75 |
| Steareth-2 | 1.00 |
| Stearic Acid | 0.75 |
| Hydrolyzed Wheat Protein | 0.10 |
| Fragrance | 1.1 |
| Polyquaternium-22 | 1.0 |
| Amodimethicone | 2.50 |
| Tetrasodium Etidronate | 0.36 |
| Thioglycolic acid | 6.74 |
| Isopropanolamine | 2.00 |
| Ammonium Bicarbonate | 1.00 |

The pH of Example Formulation 6 is preferably approximately 8.75.

### Example 2

### Consumer Study Evaluation

Of the sixty consumers that were provided a product kit made up of Example Formulations 3, 4, 5 and 6 (smoothing kit), a test panel comprised of seven consumer panelists with different hair types and length was formed. The smoothing kits along with directions for use of the smoothing kits were provided to each consumer panelist. The directions provided guidance in accordance with the methods of the present invention, and included instructions regarding shampooing the hair to be treated and towel blotting dry, applying a hair softening composition in accordance with either Example Formulation 5 or Example Formulation 6 depending upon the type of hair to be treated of the consumer panelist, allowing the hair softening composition to process for approximately 10-30 minutes at ambient temperatures, rinsing the hair softening composition from the hair with luke-warm water, blotting dry and combing, preparing a keratin mixture of one part Example Formulation 3 and three parts Example Formulation 4, applying the keratin mixture to the hair and processing for approximately 15 minutes, combing the hair and blotting the excess keratin mixture from the hair, flat ironing the treated hair between 400-450° F., and waiting approximately 24 hours to shampoo and/or condition the treated hair.

All consumer panelists were assisted by stylists with the shampooing and the water rinsing steps of the treatment process only. All other steps of the treatment process were carried out by the consumers.

The surveys were filled out personally by each panelist before and after the treatment process. The panelists have returned after a week of the treatment process for analyzing all performance attributes of the treatment process for smoothing and revitalizing the hair. At this check a third survey on the performance attributes was filled out by the consumer or by a chemist. The specific attributes that were verified included curl reduction, smoothness/frizz reduction and effects on their daily styling grooming of hair. The data of the survey was tabulated in spreadsheets and analyzed further.

The consumer panelists' hair length ranged from long (beyond shoulder) to medium (above shoulder) and wavy to curly with fine, medium, coarse texture. Four consumer panelists had permanent color treated hair and three consumer panelists had virgin untreated hair. All performance attributes curl, frizz, shine and softness/smoothness were evaluated on a scale rating below of 0-100% before and after the service.

SCALE RATING

| | | | Somewhat | Somewhat | | Very |
|---|---|---|---|---|---|---|
| Attributes | Very Low | Low | Low | High | High | High |
| CURL | 0-10% | 20-30% | 40-50% | 90-100% | | |
| FRIZZ | 0-10% | 0-10% | 30-40% | 50-60% | 70-80% | 90-100% |
| SHINE | 0-10% | 0-10% | 30-40% | 50-60% | 70-80% | 90-100% |
| SOFTNESS/ | 0-10% | 0-10% | 30-40% | 50-60% | 70-80% | 90-100% |
| SMOOTHNESS | | | | | | |

FIG. 1 provides the comparative survey answers of styling time in minutes before and after the treatment process according to the exemplary embodiment of the present invention on all seven consumer panelists. As shown in FIG. 1, there was a 47% to 55.56% reduction in styling time. This reduction in styling time is independent of hair length, texture and color treatment history. Based on these seven consumer panelists, the average reduction in styling time is about 51.54%±3.79, and is independent of the color treatment history, length, degree of curliness and texture of hair.

FIG. 2 provides the degree of curl reduction of the treated hair of the consumer panelists in comparison to the initial curl amount of the untreated hair. The rating or scale of the level of curl ranged from somewhat high (very curly) to somewhat low (wavy) and very low (almost straight). The scale rating of curl is given as 90-100% for somewhat high (very curly), 40-50% for somewhat low (wavy) and 0-10% for very low (almost straight). Based on these seven consumer panelists the level of curl reduction is significantly high and ranged from 90-95% or the average of 93.23%±3.98. These results do show that the treatment has yielded significantly straightening regardless of the initial degree of curly or wavy.

FIGS. 6A, 7A and 8A show the untreated hair of exemplary consumer panelists selected from the seven consumer panelists, while FIGS. 6B-6C, 7B-7C and 8B-8C show the treated hair of the same selected exemplary consumer panelists shown in FIGS. 6A, 7A and 8A. Regardless of the degree of initial curl and length of hair the overall results are consistent for all consumer panelists.

FIG. 3 shows the level of frizz reduction in comparison to the initial frizz level. The rating or level of fizz ranged from somewhat very low, somewhat low, somewhat high, high and very high. The fizz scale rating is 0-10% for very low, 20-30% for low, 30-40% for somewhat low, 50-60% for somewhat high, 70-80% for high and 90-100% for very high. Based on the before and after ratings, the average % frizz reduction is about 83%±4.43. It appears that the reduction in frizz is quite pronounced for extremely frizzy hair, since the consumer evaluation is much more easily and differentiated the effects of before and after the treatment process.

FIG. 4 shows the level of shine of the treated hair in comparison to the initial level of the untreated hair. The rating or level of shine, ranged from somewhat very low, somewhat low, somewhat high, high and very high. The shine, luster/sheen scale rating is, very low 0-10%, low 20-30%, somewhat low 30-40%, somewhat high 50-60%, high 70-80% and very high 90-100%. Based on the survey results all seven consumer panelists recognized that the treatment has improved the shine of their hair. Based on the survey responses the increase in shine is quantitated to be about 54.17%±7.85. The large variance in shine indicates the difficulty in the comparative evaluation of shine of before and after the treatment.

Based on the survey results, all consumer panelists unanimously recognized that their hair had changed in texture. All seven consumer panelists have rated their hair after the treatment to somewhat high. The change from all consumer panelists reported in softness/smoothness was rated from somewhat low before the treatment to somewhat high. Based on the scale rating of softness/smoothness for somewhat low as 30-40% and somewhat high as 50-60%, represents that the softness/smoothness has been increased by about 57.14%±0.00. This observation is consistent with high confidence since all of the consumer panelists have all identified the same change in their hair from the treatment. These attributes are characteristics of the change of texture, manageability and styling behavior of hair.

The results and analysis of the keratin treatment process study shows an improvement in the performance attributes as shown in FIG. 5. The improvement in the five attributes shown in FIG. 5 includes a reduction in curl by 93.2%, reduction in frizz by 83%, reduction in styling time by 51.54%, an increase in shine by 54.17% and an increase in softness/smoothness by 57.14%. The hair's appearance, texture, manageability is different and responds easier to daily styling and grooming. Also consumer panelists have reported that the behavior of hair under high humidity conditions is different.

Results obtained from the other 53 consumers were consistent with the results obtained from the seven consumer panelists.

### Example 3

Formulations of hair softening compositions as shown in Example Formulation 5 for normal/resistant hair, and Example Formulation 6 for color treated hair were used in combination with a keratin mixture formed from Example Formulation 3 of the keratin concentrate and Example Formulation 4 of the keratin sealer and evaluated. Directions and products kits were provided to thirty-four consumers with different hair textures, density, curl pattern and length for evaluation. The study was a single-blind home test conducted with 34 non-ethnic consumer subjects ranging in age of 23-62 years with curly/wavy hair. The panel included 61% of shoulder hair length and 39% longer than shoulder. The hair types included as 49% curly and 52% wavy with primarily medium texture of 76%, 12% coarse and 12% fine texture. The majority of the subjects (39%) were color treated, with 18% being highlighted, 18% being semi-permanent color and 21% having virgin hair with no prior chemical service.

The 34 consumer subjects were provided a product kit made up of Example Formulations 3, 4, 5 and 6 (smoothing kit) along with directions for use of the smoothing kits. The directions provided guidance in accordance with the methods of the present invention, and included instructions regarding shampooing the hair to be treated and towel blotting dry, applying a hair softening composition in accordance with either Example Formulation 5 or Example Formulation 6 depending upon the type of hair to be treated of the consumer subject, allowing the hair softening composition to process for approximately 10-30 minutes at ambient temperatures, rinsing the hair softening composition from the hair with luke-warm water, blotting dry and combing, preparing a keratin mixture of one part Example Formulation 3 and three parts Example Formulation 4, applying the keratin mixture to the hair and processing for approximately 15 minutes, combing the hair and blotting the excess keratin mixture from the hair, flat ironing the treated hair between 400-450° F., and waiting approximately 24 hours to shampoo and/or condition the treated hair.

The consumer subjects were given questionnaires to evaluate based on their perception of their hair attributes before and after use of the smoothing kits, after one, two and three months of use. The hair attributes included the styling time, amount of curl, frizz, shine, softness/smoothness and healthy look and feel.

Consumer Perception of Product Effects on the styling time (N = 34)

| | 5-15 | | | |
|---|---|---|---|---|
| Styling Time | minutes | 16-30 minutes | 31-45 minutes | 46 + minutes |
| Initial | 27% | 46% | 24% | 3% |
| After One | 55% | 39% | 6% | 0 |
| Shampoo | | | | |
| After 1 Month | 46% | 33% | 18% | 3% |
| After 2 Months | 36% | 46% | 15% | 3% |
| After 3 Months | 42% | 33% | 15% | 9% |

The consumer subjects' perception after use of the smoothing kits indicated that their styling time has been reduced by an average minimum of 35% after their first shampoo. The largest effects are noted are noted for those who have an average styling time of more than 30 minutes. The effects on reduction of styling time are still apparent after one, two and three months, where the majority (80%) of subjects had shampooed their hair 3-4 times, and 20% had shampooed their hair 1-2 times a week.

Consumer Perception of Product Effects on the Curl (N = 34)

| Amount of Curl | 0-40% | 40-100% |
|---|---|---|
| Initial | 21% | 79% |
| After One Shampoo | 100% | 0% |
| After 1 Month | 100% | 0% |
| After 2 Months | 97% | 3% |
| After 3 Months | 58% | 42% |

The consumer subjects' perception was that their curl/wave has been reduced by 100% initially, and after the first shampoo. This reduction in curl is quite significant regardless of how curly initially the hair is. The effects on of curl reduction are still apparent after one, two and three months where the majority (80%) of subjects had shampooed their hair 3-4 times, and 20% had shampooed their hair 1-2 times a week.

Consumer Perception of Product Effects on Frizz (N = 34)

| Amount of Frizz | 0-40% | 40-100% |
|---|---|---|
| Initial | 39% | 61% |
| After One Shampoo | 100% | 0% |
| After 1 Month | 100% | 0% |
| After 2 Months | 97% | 3% |
| After 3 Months | 64% | 36% |

The consumer subjects' perception was that their frizz has been reduced by 100% initially, and after their first shampoo. The effects on of frizz reduction are still apparent after one, two and three months where the majority (80%) of subjects had shampooed their hair 3-4 times, and 20% had shampooed their hair 1-2 times a week.

Consumer Perception of Product Effects on Shine (N = 34)

| Level of shine | 0-40% | 40-100% |
|---|---|---|
| Initial | 64% | 36% |
| After One Shampoo | 0% | 100% |
| After 1 Month | 0% | 100% |
| After 2 Months | 6% | 94% |
| After 3 Months | 27% | 73% |

The consumer subjects' perception was that their shine has been increased by 100% initially, and after their first shampoo. The effects on of increase in shine are still apparent after one, two and three months where the majority (80%) of subjects had shampooed their hair 3-4 times, and 20% had shampooed their hair 1-2 times a week.

Consumer Perception of Product Effects on Softness/Smoothness
(N = 34)

| Level of Softness/Smoothness | 0-40% | 40-100% |
|---|---|---|
| Initial * | 46% | 55% |
| After One Shampoo | 0% | 100% |
| After 1 Month | 0% | 100% |
| After 2 Months | 6% | 94% |
| After 3 Months | 24% | 76% |

The consumer subjects' perception was that their softness/smoothness has been increased by 100% initially, and after their first shampoo. The effects of increase in their softness are still apparent after one, two and three months.

This single-blind product testing of the smoothing kit comprised of Example Formulations 3, 4, 5 and 6 shows that the effects are long lasting, i.e. at least three months for the majority of the consumer subjects. It is conceivable that the longevity it may be beyond 90 days for certain hair types, and less for certain other hair types.

Other exemplary hair softening compositions according to the present invention, and may be used in accordance with the methods of the present invention are presented below.

### Example 4

EXAMPLE FORMULATION 7

| CLEAR GEL HAIR SOFTENING COMPOSITION FOR | |
|---|---|
| NORMAL/RESISTANT HAIR | w/w % |
| Water | 57.92 |
| Sodium Borate | 1.00 |
| Tetrasodium Etidronate | 0.36 |
| Ethanolamine | 5.4 |
| Sucrose | 7 |
| Cysteine HCl | 3.5 |
| Cysteamine HCl (75%) | 12.67 |
| Acrylates Copolymer | 6.50 |
| Laureth-23 | 4 |
| Fragrance (Parfum) | 0.65 |
| PEG-12 Dimethicone | 1 |

The pH of Example Formulation 7 is preferably 8.1-8.5.

### Example 5

EXAMPLE FORMULATION 8

| HAIR SOFTENING COMPOSITION FOR BLEACHED | |
|---|---|
| OR DELICATE HAIR | w/w % |
| Water | 68.81 |
| Polyacrylate Crosspolymer-6 | 0.5 |
| Ammonium Citrate | 1 |
| Cetearyl Alcohol | 2 |
| Cetyl Alcohol | 1 |
| Steareth-21 | 0.75 |
| Steareth-2 | 1 |
| Stearic Acid | 0.75 |
| Propylene Glycol | 1.74 |
| Urea | 3 |
| Isopropyl Alcohol | 1 |
| Sucrose | 0.5 |
| Pentasodium Pentetate | 0.2 |
| Fragrance/Parfum | 0.5 |
| Ammonium Bisulfite | 15 |
| Isopropanolamine | 2.5 |
| Sodium Polyacrylate (and) Hydrogenated Polydecene | 0.75 |

The pH of Example Formulation 8 is preferably 6.5-7.0

It will thus be seen that the objects set forth above, among those made apparent from the preceding description, are efficiently attained and, since certain changes may be made in the above invention without departing from the scope of this invention, it is intended that all matter contained in this disclosure or shown in the accompanying drawings, shall be interpreted, as illustrative and not in a limiting sense. It is to be understood that all of the present figures, and the accompanying narrative discussions of corresponding embodiments, do not purport to be completely rigorous treatments of the invention under consideration. It is to be understood that the above-described arrangements are only illustrative of the application of the principles of the present invention. Numerous modifications and alternative arrangements may be devised by those skilled in the art without departing from the scope of the present invention.

1. A method of treating keratin fibers, comprising:
applying a softening composition comprising at least one reducing agent to the keratin fibers,
applying a keratin mixture comprising at least one hydrolyzed keratin and at least one oxidizing agent to the keratin fibers, and
heating the keratin fibers to at least 400° F.

2. The method according to claim 1, wherein the at least one reducing agent comprises Thioglycolic acid, Thiolactic acid, 3-mercaptoproprionic acid, Thiomalic acid, Glyceryl Mono Thioglycolate, Cysteine, Thioglycerol (3-Mercaptopropane-1,2-diol), Cysteamine, sulfites, bisulfites or combinations thereof.

3. The method according to claim 1, wherein the at least one reducing agent comprises about 6-16% of the softening composition based on the entire weight of the softening composition.

4. The method according to claim 1, wherein the keratin mixture is comprised of approximately one part keratin concentrate composition comprising the at least one hydrolyzed keratin and approximately three parts keratin sealer composition comprising the at least one oxidizing agent.

5. The method according to claim 4, wherein the keratin concentrate composition further comprises about 0.1-0.5% of at least one thickening agent, about 2-15% of at least one quaternized hydrolyzed keratin, and about 1-6% at least one silicone cystine and/or silicone cysteine copolymer based on the entire weight of the keratin concentrate composition.

6. The method according to claim 4, wherein the at least one hydrolyzed keratin comprises about 4-30% of the keratin concentrate composition based on the entire weight of the keratin concentrate composition.

7. The method according to claim 1, wherein the oxidizing agent comprises hydrogen peroxide, potassium perborate, potassium bromated or combinations thereof.

8. The method according to claim 1, wherein the keratin fibers comprise hair.

9. The method according to claim 1, wherein the softening composition is applied to the keratin fibers for 10 to 30 minutes.

10. The method according to claim 1, further comprising rinsing at least part of the hair softening composition from the keratin fibers prior to applying the keratin mixture.

11. A kit for treating keratin fibers, comprising:
a softening composition comprising at least one reducing agent;
a keratin concentrate composition comprising at least one hydrolyzed keratin; and a keratin sealer composition comprising at least one oxidizing agent; wherein the keratin concentrate composition is configured for mixing with the keratin sealer composition to form a keratin mixture.

12. The kit according to claim 11, wherein the at least one reducing agent comprises Thioglycolic acid, Thiolactic acid, 3-mercaptoproprionic acid, Thiomalic acid, Glyceryl Mono Thioglycolate, Cysteine, Thioglycerol (3-Mercaptopropane-1,2-diol), Cysteamine, sulfites, bisulfites or combinations thereof.

13. The kit according to claim 11, wherein the at least one reducing agent comprises approximately 6-16% of the softening composition based on the entire weight of the softening composition.

14. The kit according to claim 11, wherein the keratin mixture is comprised of approximately one part of the keratin concentrate composition and approximately three parts of the keratin sealer composition.

15. The kit according to claim 11, wherein the keratin concentrate composition further comprises about 0.1-0.5% of at least one thickening agent, about 2-15% of at least one quaternized hydrolyzed keratin, and about 1-6% at least one silicone cystine and/or silicone cysteine copolymer based on the entire weight of the keratin concentrate composition.

16. The method according to claim 11, wherein the at least one hydrolyzed keratin comprises about 4-30% of the keratin concentrate composition based on the entire weight of the keratin concentrate composition.

17. The kit according to claim 11, wherein the oxidizing agent comprises hydrogen peroxide, potassium perborate, potassium bromated or combinations thereof.

18. The kit according to claim 15, wherein the at least one silicone cystine and/or silicon cysteine copolymer comprises cystine bis-PG-propyl silanetriol.

19. The kit according to claim 11, wherein the softening composition is configured to form at least one active site on the keratin fibers; and wherein the keratin mixture is configured to fix or cross-link the at least one hydrolyzed keratin to the at least one active site.

20. The kit according to claim 11, wherein the reducing agent comprises at least one acylated derivative of Cysteine, Thioglycerol or Cysteamine.
